(19) United States
(12) Patent Application Publication
(10) Pub. No.: US 2006/0034906 A1
(43) Pub. Date: Feb. 16, 2006

(54) Treatment of lung diseases and pre-lung disease conditions

(75) Inventors: Lawrence T. Boni, Monmouth Junction, NJ (US); Walter Perkins, Pennington, NJ (US); Roman Perez-Soler, New York, NY (US); Frank G. Pilkiewicz, Princeton Junction, NJ (US)

Correspondence Address: FOLEY HOAG, LLP;PATENT GROUP, WORLD TRADE CENTER WEST, 155 SEAPORT BLVD, BOSTON, MA, 02110, US

(73) Assignee: Transave, Inc., Monmouth, NJ (US)

(21) Appl. No.: 11/135,625

(22) Filed: May 23, 2005

Related U.S. Application Data
(60) Provisional application No. 60/573,088, filed on May 21, 2004.

(51) Int. Cl. A61K 33/24; A61K 9/127; A61K 31/56

(52) U.S. Cl. 424/450; 424/649; 514/171

(57) ABSTRACT

In part, the present invention relates to a method of treating lung diseases and pre-lung disease conditions such as precancerous lesions comprising administering to a patient in need a therapeutic agent comprising lipid composition. The present invention also relates to an inhalation device for administering lipid complexes comprising therapeutic agents. The inhalation device may be disposable. In one embodiment, the lung diseases pretreated by the methods of the present invention are those diseases associated with tobacco related products. The present invention also relates to a method of preparing liposomes by an infusion method that yields high entrapment percentages.

### RELATED APPLICATIONS

This application claims the benefit of U.S. Provisional Patent Application Ser. No. 60/573,088, filed May 21, 2004; the entirety of which is incorporated by reference.

### BACKGROUND OF THE INVENTION

The present invention relates to a method for treating lung diseases and pre-lung disease conditions (e.g. precancerous lesions) by delivering a therapeutically effective amount of a lipid composition comprising a therapeutic agent (e.g., cisplatin (cis-diamine-dichloroplatinum (II))) to a patient's respiratory tract. In particular, the present invention relates to the treatment and pretreatment of lung diseases as a consequence of smoking tobacco related products. The method allows for early treatment of precancerous conditions and for more frequent treatment cycles without the attendant side effects (e.g., nephrotoxicity, bone marrow toxicity) common to systemic administration of many cancer cytotoxic agents.

Typically, chemotherapeutic treatment of lung cancers includes systemic administration of chemotherapeutic agents, e.g., cytotoxic agents, to the patients. Often such administration, e.g., intravenous administration, is associated with several adverse side effects including nephrotoxicity and bone marrow toxicity. For instance, systemic administration of cisplatin one of the more effective anti-tumor agents used in the systemic treatment of lung cancers, is often burdened by symptoms such as nephrotoxicity in the patient. The nephrotoxicity limits the frequency in which clinicians can administer cisplatin to the patient. In fact, successive treatment cycles of cisplatin typically require three weeks or more between treatment cycles to prevent blood levels of cisplatin from reaching those correlated with nephrotoxicity. Since chemotherapeutic regimens typically require five or more treatment cycles, the delay between treatment cycles lengthens the time needed for the overall chemotherapeutic regimen. The prolonged time periods for systemic administration of cisplatin lead to increased patient discomfort and inconvenience, and may lead to decreased patient compliance.

Inhalation therapeutics are an attractive alternative to injectables for treating lung disease because they provide higher drug levels in the lung, ease of use, and reduced cost. However, current inhalation therapies have significant disadvantages which have limited their use in this area such as: 1) short term therapeutic effects due to rapid clearance of the drug from the lung, requiring frequent administration of the drug, 2) no enhanced targeting to diseased cells, 3) no protection from in vivo degradation in the lung.

Accordingly, new methods for pretreating patients in the early stages of lung disease by inhalation administration of therapeutic agents are desirable. Such methods preferably also overcome the rapid clearance of therapeutic agent from the lung that typically plague inhalation administration of therapeutic agents.

### SUMMARY OF THE INVENTION

The present invention utilizes a sustained release lipid inhalation targeting technology to address disadvantages associated with current inhalation treatments and broadens the potential of inhalation therapy by using lipids, lipid complexes and liposomes engineered to optimize the sustained release and targeting of drugs to the lungs' microenvironment, and protect the drug from in vivo degradation. Lipid based delivery systems of the present invention can utilize traditional off-patent inhalation devices, and have the ability to be administered for inhalation either as a nebulized spray or a dry powder. The use of lipid delivery systems to improve the usage and the therapeutic index of a drug has had success in the development of injectable drugs.

In part, the invention comprises a hand held devise, envisioned in one embodiment to be similar to a nicotine inhaler (e.g. Nicotrol Inhaler) that contains a lipid formulation of the present invention. The lipid formulation comprises a therapeutic agent. In certain embodiments, the formulation may be in a liquid or powder form. In further embodiments, the device will be adjustable such that upon inhaling, a calculated amount of the lipid formulation of the present invention will be delivered. The present invention may be used in the chemoprevention of diseases smokers are susceptible to (e.g., lung cancer for smokers prior to cellular changes), prophylactic treatment to high risk groups (e.g., gene therapy or antineoplastics to smokers upon first indication of cellular change) and disease stage treatment of the disease (e.g., antineoplastics for smokers with cancer or antibacterials for smokers with infections). In a further embodiment, the inhalation device is disposable.

### DETAILED DESCRIPTION OF THE INVENTION

Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The term “bioavailable” is art-recognized and refers to a form of the subject invention that allows for it, or a portion of the amount administered, to be absorbed by, incorporated to, or otherwise physiologically available to a subject or patient to whom it is administered.

The phrase “effective amount” refers to that amount of a substance that produces some desired local or systemic effect at a reasonable benefit/risk ratio applicable to any treatment. The effective amount of such substance will vary depending upon the subject and disease condition being treated, the weight and age of the subject, the severity of the disease condition, the manner of administration and the like, which can readily be determined by one of ordinary skill in the art.

A “patient,” “subject” or “host” may be a human or non-human animal.

The term “pharmaceutically acceptable salts” is art-recognized and refers to the relatively non-toxic, inorganic and organic acid addition salts of compounds, including, for example, those contained in compositions of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be acceptable in the sense of being compatible with the subject composition and its components and not injurious to the patient. Some examples of materials which may serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The phrase “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art.

The term “treating” is art-recognized and refers to curing as well as ameliorating at least one symptom of any condition or disease.

Lipids

The lipids used in the lipid compositions of the present invention can be synthetic, semi-synthetic or naturally-occurring lipids, and typically include phospholipids and steroids, which include, for example, sterols. In terms of phosholipids, they could include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC, a major constituent of naturally-occurring lung surfactant. Other examples include dimyristoylphosphatidycholine (DMPC) and dimyristoylphosphatidylglycerol (DMPG), dipalmitoylphosphatidylglycerol (DPPG) distearoylphosphatidylcholine (DSPC) and distearoylphosphatidylglycerol (DSPG), dioleylphosphatidyl-ethanolarnine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

The steroids may include, for example, sterols. The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate.

In a preferred embodiment of the invention the lipid composition contains 50 to 100 mol % DPPC and 0 to 50 mol % cholesterol. More preferably, the lipid complex contains 50 to 65 mol % DPPC and 35 to 50 mol % cholesterol.

Methods of Preparing the Lipid Compositions

The lipid composition is preferably formed as described in co-pending U.S. patent application Ser. No. 10/634,144, filed Aug. 4, 2003, which is hereby incorporated by reference in its entirety. Briefly, the lipid complex can be formed by mixing the therapeutic agent (e.g. cisplatin) with an appropriate lipid dissolved or suspended in a solvent (e.g., ethanol) and subjecting the mixture to one or more cycles have two separate temperatures. The process procuces a therapeutic agent comprising lipid complex believed to be in the form of an active compound aggregate.

The process includes combining a therapeutic agent with a hydrophobic matrix carrying system (lipid/solvent mixture) and cycling the solution between a warmer and a cooler temperature. Preferably, the cycling is performed more than one time. More preferably, the step is performed two or more times, or three or more times. The cooler temperature portion of cycle can, for example, use a temperature from about −25° C. and about 25° C. More preferably, the step uses a temperature from about −5 and about 5° C. or between about 1 and about 5° C. For manufacturing convenience, and to be sure the desired temperature is established, the cooler and warmer steps can be maintained for a period of time, such as approximately from about 5 to about 300 minutes or about 30 to about 60 minutes. The step of warming includes warming the reaction vessel to from about 4 and about 70° C. More preferably, the step of warming comprises heating the reaction vessel to from about 45 to about 55° C. The above temperature ranges are particularly preferred for use with lipid compositions containing predominantly dipalmitoylphosphatidycholine (DPPC) and cholesterol.

Another way to consider the temperature cycling is in terms of the temperature differential between the warmer and the cooler steps of the cycle. This temperature differential can be, for example, about 25° C. or more, such as a differential from about 25 to about 70° C., preferably a differential from about 40 to about 55° C. The temperatures of the cooler and higher temperature steps are selected on the basis of increasing entrapment of therapeutic agents. Without being limited to theory, it is believed that it is useful to select an upper temperature effective to substantially increase the solubility of active platinum compound in the processed mixture. Preferably, the warming step temperature is about 50° C. or higher. The temperatures can also be selected to be below and above the transition temperature for a lipid in the lipid composition.

The temperatures appropriate for the method describe above may, in some cases, vary with the lipid composition used in the method, as can be determined by ordinary experimentation.

Therapeutic Agents

Some specific examples of therapeutic agents that can be present in the compositions of the inhalation system and the uses of the system in the treatment of disease include: sulfonamide, such as sulfonamide, sulfamethoxazole and sulfacetamide; trimethoprim, particularly in combination with sulfamethoxazole; a quinoline such as norfloxacin and ciprofloxacin; a beta-lactam compound including a penicillin such as penicillin G, penicillin V, ampicillin, amoxicillin, and piperacillin, a cephalosporin such as cephalosporin C, cephalothin, cefoxitin and ceftazidime, other beta-lactam antibiotics such as imipenem, and aztreonam; a beta lactamase inhibitor such as clavulanic acid; an aminoglycoside such as gentamycin, amikacin, tobramycin, neomycin, kanamycin and netilmicin; a tetracycine such as chlortetracycline and doxycycline; chloramphenicol; a macrolide such as erythromycin; or miscellaneous antibiotics such as clindamycin, a polymyxin, and bacitracin for anti-bacterial, and in some cases antifingal, infections; a polyene antibiotic such as amphotericin B, nystatin, and hamycin; flucytosine; an imidazole or a triazole such as ketoconazole, miconazole, itraconazole and fluconazole; griseofulvin for anti-Fungal diseases such as aspergillosis, candidaisis or histoplasmosis; zidovudine, acyclovir, ganciclovir, vidarabine, idoxuridine, trifluridine, an interferon (e.g, interferon alpha-2a or interferon alpha-2b) and ribavirin for anti-viral disease; aspirin, phenylbutazone, phenacetin, acetaminophen, ibuprofen, indomethacin, sulindac, piroxicam, diclofenac; gold and steroidal anti-inflammatories for inflammatory diseases such as arthritis; an ACE inhibitor such as captopril, enalapril, and lisinopril; the organo nitrates such as amyl nitrite, nitroglycerin and isosorbide dinitrate; the calcium channel blockers such as diltiazem, nifedipine and verapamil; the beta adrenegic antagonists such as propranolol for cardiovascular disease; a diuretic such as a thiazide; e.g., benzothiadiazine or a loop diuretic such as furosemide; a sympatholytic agent such as methyldopa, clonidine, gunabenz, guanaethidine and reserpine; a vasodilator such as hydalazine and minoxidil; a calcium channel blocker such as verapimil; an ACE inhibitor such as captopril for the treatment of hypertension; quinidine, procainamide, lidocaine, encainide, propranolol, esmolol, bretylium, verapimil and diltiazem for the treatment of cardiac arrhythmia; lovostatin, lipitor, clofibrate, cholestryamine, probucol, and nicotinic acid for the treatment of hypolipoproteinernias; an anthracycline such as doxorubicin, daunorubicin and idarubicin; a covalent DNA binding compound, a covalent DNA binding compound and a platinum compound such as cisplatin and carboplatin; a folate antagonist such as methotrexate and trimetrexate; an antimetabolite and a pyrimidine antagonist such as fluorouracil, 5-fluorouracil and fluorodeoxyuridine; an antimetabolite and a purine antagonist such as mercaptopurine, 6-mercaptopurine and thioguanine; an antimetabolite and a sugar modified analog such as cytarabine and fludarabine; an antimetabolite and a ribonucleotide reductase inhibitor such as hydoxyurea; a covalent DNA binding compound and a nitrogen mustard compound such as cyclophosphamide and ifosfamide; a covalent DNA binding compound and an alkane sulfonate such as busulfane; a nitrosourea such as carmustine; a covalent DNA binding compound and a methylating agent such as procarbazine; a covalent DNA binding compound and an aziridine such as mitomycin; a non covalent DNA binding compound; a non covalent DNA binding compound such as mitoxantrone and, bleomycin; an inhibitor of chromatin function and a topoisomerase inhibitor such as etoposide, teniposide, camptothecin and topotecan; an inhibitor of chromatin function and a microtubule inhibitor such as the vinca alkaloids including vincristine, vinblastin, vindisine, and paclitaxel, taxotere or another taxane; a compound affecting endocrine function such as prednisone, prednisolone, tamoxifen, leuprolide, ethinyl estradiol, an antibody such as herceptin; a gene such as the p-53 gene, the p 16 gene, the MIT gene, and the gene E-cadherin; a cytokine such as the interleukins, particularly, IL-1, IL-2, IL-4, IL-6, IL-8 and IL-12, the tumor necrosis factors such as tumor necrosis factor-alpha and tumor necrosis factor-beta, the colony stimulating factors such as granulocyte colony stimulating factor (G-CSF), macrophage colony stimulating factor (M-CSF) and, granulocyte macrophage colony stimulating factor (GM-CSF) an interferon such as interferon-alpha, interferon-beta 1, interferon-beta 2, and interferon-gamma; all-trans retinoic acid or another retinoid for the treatment of cancer; an immunosupressive agent such as: cyclosporine, an immune globulin, and sulfasazine, methoxsalen and thalidoimide; insulin and glucogon for diabetes; calcitonin and sodium alendronate for treatment of osteoporosis, hypercalcemia and Paget's Disease; morphine and related opioids; meperidine or a congener; methadone or a congener; an opioid antagonist such as nalorphine; a centrally active antitussive agent such as dexthromethrophan; tetrahydrocannabinol or marinol, lidocaine and bupivicaine for pain management; chloropromazine, prochlorperazine; a cannabinoid such as tetrahydrocannabinol, a butyrophenone such as droperidol; a benzamide such as metoclopramide for the treatment of nausea and vomiting; heparin, coumarin, streptokinase, tissue plasminogen activator factor (t-PA) as anticoagulant, antithrombolytic or antiplatelet drugs; heparin, sulfasalazine, nicotine and adrenocortical steroids and tumor necrosis factor-alpha for the treatment of inflammatory bowel disease; nicotine for the treatment of smoking addiction; growth hormone, luetinizing hormone, corticotropin, and somatotropin for hormonal therapy; and adrenaline for general anaphylaxis.

Further therapeutic agents that can be present in the compositions of the inhalation system and the uses of the system in the treatment of disease include: a methylxanthine such as theophylline; cromolyn; a beta-adrenginic agonist such as albuterol and tetrabutaline; a anticholinergic alkaloid such as atropine and ipatropium bromide; adrenocortical steroids such as predisone, beclomethasone and dexamethasone for asthma or inflammatory disease; the anti-bacterial and antifungal agents listed above for anti-bacterial and anti-fungal infections in patients with lung disease (these are the specific diseases listed above in what lung disease includes), in particular this includes the use of aminoglycosides (e.g., amikacin, tobramycin and gentamycin), polymyxins (e.g., polymyxin E, colistin), carboxycillin (ticarcillin) and monobactams for the treatment of gram-negative anti-bacterial infections, for example, in cystic fibrosis patients, for the treatment of gram negative infections of patients with tuberculosis, for the treatment of gram negative infections in patients with chronic bronchitis and bronchiectasis, and for the treatment of gram negative infections in generally immuno-compromised patients; the use of pentamidine for the treatment of patients (e.g., HIV/AIDS patients) with Pneumocytis carinii infections; the use of a polyene antibiotic such as amphotericin B, nyststin, and hamycin; flucytosine; an imidazole or a triazole such as ketoconazole, miconazole, itraconazole and fluconazole; griseofulvin for the treatment of such fungal infections as aspergillosis, candidiasis and histoplasmosis, particularly those originating or diseminating to the lungs; the use of the corticosteroids and other steroids as listed above, as well as nonsteroidal anti-inflammatory drugs for the treatment of anti-inflammatory conditions in patients with lung disease (these are the specific diseases listed above in what lung disease includes); DNase, amiloride, CFTRcDNA in the treatment of cystic fibrosis; alpha-1-antitrypsin and alpha-1-antitrypsin cDNA for the treatment of emphysema; an aminoglycoside such as amikacin, tobramycin or gentamycin, isoniazid, ethambutol, rifampin and its analogs for the treatment of tuberculosis or mycobacterium infections; ribavirin for the treatment of respiratory synctial virus; the use of the anticancer agents listed above for lung cancer in particular cisplatin, carboplatin, and taxanes such as paclitaxel, and the taxanes, camptothecin, topotecin, and other camptothecins, herceptin, the p-53 gene and IL-2. In addition, pharmaceutical therapeutic agents such as Tarceva and Iressa may also be used.

The pharmaceutical formulation of the inhalation system of the present invention may contain more than one therapeutic agent (e.g., two therapeutic agents for a synergistic effect).

Cisplatin as the Active Agent

In aqueous solution, cisplatin forms large crystalline aggregates with a crystal diameter of greater than a few microns. In the presence of an amphipathic matrix system, such as a lipid bilayer, cisplatin complexes with the lipid. For example, the complexes may be formed in the hydrocarbon core region of a lipid bilayer. During the warming cycle of the process, it is believed that cisplatin is returned to solution at a greater rate in aqueous regions of the process mixture than in the bilayers. As a result of applying more than one cool/warm cycle, cisplatin accumulates further in the lipid bilayers. Without limiting the invention to the proposed theory, experimentation indicates that the cisplatin complexes cause the immediate surroundings of the interfacial bilayer region to be more hydrophobic and compact. This results in a high level of entrapment of active platinum compound as cooling and warming cycles are repeated.

The formulation has a markedly high entrapment percentage of cisplatin. The entrapment has been shown, in some cases, to reach upto about 20, 30, 40, 50, 60, 70, 80, or about 90%. This amount is far higher than the most efficient entrapment expected from a conventional aqueous entrapment which is approximately 2-10% entrapment.

Experimental results strongly indicate that encapsulation was achieved predominantly by capturing cisplatin during formation of liposomal vesicles. The results further indicate the physical state of cisplatin to be solid (aggregates) or lipid bound since the concentration of cisplatin is much higher than the solubility limit. Results further indicate that process does not require freezing the compositions, but that cooling to temperature higher than freezing can produce superior results. Results further indicated that an entrapment efficiency achieved by 3 cycles was similar to that achieved by 6 cycles of cooling and warming cycles, which indicated that 3 cycles of temperature treatment was sufficient to achieve highly preferred levels of entrapment.

Results further indicate that the process can be scaled-up while increasing process efficiency in entrapping cisplatin. Thus, the invention further provides processes that are conducted to provide an amount adapted for total administration (in appropriate smaller volume increments) of about 200 or more mLs, about 400 or more mLs, or about 800 or more mLs. All else being the same, it is believed that the larger production volumes generally achieve increased efficiency over smaller scale processes. While such volume is that appropriate for administration, it will be recognized that the volume can be reduced for storage.

Results further indicate that the lipid-complexed cisplatin made by this method can retain entrapped cisplatin with minimal leakage for over one year. This is a further demonstration of the uniqueness in the formulation, indicating that the cisplatin is bound within the liposome structure and not free to readily leak out.

Methods of Administration

Generally, the lipid formulations of the present invention may be administered parenterally or by inhalation. Parenteral routes of administration involve injections into various compartments of the body. Parenteral routes include intravenous (iv), i.e. administration directly into the vascular system through a vein; intra-arterial (ia), i.e. administration directly into the vascular system through an artery; intraperitoneal (ip), i.e. administration into the lung cavity; subcutaneous (sc), i.e. administration under the skin; intramuscular (im), i.e. administration into a muscle; and intradermal (id), i.e. administration between layers of skin. The parenteral route is preferred over oral ones in many occurrences. For example, when the drug to be administered would partially or totally degrade in the gastrointestinal tract, parenteral administration is preferred. Similarly, where there is need for rapid response in emergency cases, parenteral administration is usually preferred over oral.

Inhalation is generally preferred for the treatment of lung diseases or pre-lung disease conditions and involves a delivery device. The inhalation delivery device of the inhalation system can be a nebulizer, a metered dose inhaler (MDI) or a dry powder inhaler (DPI). The device can contain and be used to deliver a single dose of the lipid compositions or the device can contain and be used to deliver multi-doses of the lipid compositions of the present invention. In another embodiment, the nebulizer is envisioned to be disposable.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically (including, for example, a vibrating porous membrane). The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device can consist of a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer is employed to ensure the sizing of aqueous droplets containing the drug-lipid particles is optimal for positioning of the particle within, for example, the lungs. Typical droplet sizes for the nebulized lipid composition are from about to about 5 microns.

For use with the nebulizer, the lipid composition preferably contains an aqueous component. Typically there is at least about 80% by weight and preferably, at least about 90% by weight of the aqueous component in the lipid composition to be administered with a nebulizer. The aqueous component may include for example, saline. In addition, the aqueous component may include up to about 20% by weight of an aqueous compatible solvent such as ethanol.

Total administration time using a nebulizer will depend on the flow rate and the concentration of the cisplatin in the lipid composition. Variation of the total administration time is within the purview of those of ordinary skill in the art. Generally, the flow rate of the nebulizer will be at least about 0.15 mL/min, for example, a flow rate of about 0.2 mL/min is typical. By way of example, administration of a dose of about 24 mg/m² of cisplatin using a lipid composition having a concentration of about 1 mg/mL of cisplatin would be about 4 hours (assuming a patient's body surface area is about 2 m²). This administration time may, for example, be split into two administration sessions given over the course of one or two days to complete one treatment cycle.

In alternative embodiments, a metered dose inhalator (MDI) can be employed as the inhalation delivery device of the inhalation system. This device is pressurized (PMDI) and its basic structure consists of a metering valve, an actuator and a container. A propellant is used to discharge the formulation from the device. The composition can consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. Traditional chloroflourocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid based formulation. To insure accuracy of dosing, the delivery of the formulation may be programmed via a microprocessor to occur at a certain point in the inhalation cycle. The MDI may be portable and hand held.

In another alternative embodiment, a dry powder inhalator (DPI) can be used as the inhalation delivery device of the inhalation system. This device's basic design consists of a metering system, a powdered composition and a method to disperse the composition. Forces like rotation and vibration can be used to disperse the composition. The metering and dispersion systems may be mechanically or electrically driven and may be microprocessor programmable. The device may be portable and hand held. The inhalator may be multi or single dose in design and use such options as hard gelatin capsules, and blister packages for accurate unit doses. The composition can be dispersed from the device by passive inhalation; i.e., the patient's own inspiratory effort, or an active dispersion system may be employed. The dry powder of the composition can be sized via processes such as jet milling, spray dying and supercritical fluid manufacture. Acceptable excipients such as the sugars mannitol and maltose may be used in the preparation of the powdered formulations. These are particularly important in the preparation of freeze dried liposomes and lipid complexes. These sugars help in maintaining the liposome's physical characteristics during freeze drying and minimizing their aggregation when they are administered by inhalation. The hydroxyl groups of the sugar may help the vesicles maintain their tertiary hydrated state and help minimize particle aggregation.

The inventive method is particularly well-suited for the pre-treatment and treatment of lung cancers. In addition, both primary and metastatic lung cancers are excellent candidates for the method of the invention.

Dosages and Treatment

Administration of the compositions of the present invention will be in an amount sufficient to achieve a therapeutic effect as recognized by one of ordinary skill in the art.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

In certain embodiments, the dosage of the subject compounds will generally be in the range of about 1.5 mg/m² to about 80 mg/m². In another embodiment the dosage may be in the range of about 3.0 mg/m² to about 80 mg/m². In another embodiment the dosage may be in the range of about 6.0 mg/m² to about 80 mg/m². In another embodiment the dosage may be in the range of about 12.0 mg/m² to about 80 Mg/m². In another embodiment the dosage may be in the range of about 24.0 mg/m² to about 80 mg/m². In another embodiment the dosage may be in the range of about 30.0 mg/m² to about 80 mg/m². In another embodiment the dosage may be in the range of about 30.0 mg/m² to about 80 mg/m². In another embodiment the dosage may be in the range of about 36.0 mg/m² to about 80 mg/m². In another embodiment the dosage may be in the range of about 40.0 m/m² to about 80 mg/m². In another embodiment the dosage may be in the range of about 48.0 mg/m² to about 80 mg/m². In another embodiment the dosage may be in the range of about 60.0 mg/m² to about 80 mg/m².

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

Treatment may be described in terms of treatment cycles. Treatment cycles, as used herein, describe the frequency of treatments and, in that sense, the time between treatments. For example, a treatment cycle of 3 weeks means that the patient undergoes treatment once every 3 weeks. A treatment cycle of 2 weeks means that the patient undergoes treatment once every 2 weeks. A treatment cycle of 1 week means that the patient undergoes treatment once every week.

The actual treatment itself may be described in terms hours, days, every other day, every other two days . . . etc. For example, treatment may include daily treatments for anywhere from 1 to 7 days. Treatment, alternatively, may include treatments every other day for anywhere from 1 to 14 days, or from 1 to 7 days. The amount of variations possible are limited only by the recommended regiment of one of ordinary skill in the art. For example, treatment may be daily for anywhere from 1 to 7 days, and such a treatment may be administered on a weekly time cycle, which means that after undergoing such treatment, the patient will have a one week break before undergoing the same treatment, or a modified treatment (for instance, it is envisioned by the inventors that initial treatment may include high dosages and frequency, but that ongoing treatments, as the patient improves, are reduced).

The treatment methods may also be described in terms of the actual administration time, i.e. the time that the patient is undergoing the actual treatment. Generally, the less time the better because of the convenience to the patient and the less time the patient may have to spend in a hospital. The actual treatment time may be over several hours, e.g. anywhere from 3 to 6 hours, or it may be just 2 hours or 1 hour, or less than 1 hour. For example, actual treatment time may be as low as 20 minutes or less.

The use of the subject compositions may reduce the required dosage for any individual agent contained in the compositions (e.g., the steroidal anti inflammatory drug) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

In general, the doses of an active agent will be chosen by a physician based on the age, physical condition, weight and other factors known in the medical arts.

Efficacy of Treatment

The efficacy of treatment with the subject compositions may be determined in a number of fashions known to those of skill in the art.

In one exemplary method, the median rate of decrease in tumor or lesion size from treatment with a subject composition may be compared to other forms of treatment with the particular therapeutic agent contained in the subject composition, or with other therapeutic agents. The decrease in tumor or lesion size for treatment with a subject composition as compared to treatment with another method may be 10, 25, 50, 75, 100, 150, 200, 300, 400% greater or even more. The period of time for observing any such decrease may be about 1, 3, 5, 10, 15, 30, 60 or 90 or more hours. The comparison may be made against treatment with the particular therapeutic agent contained in the subject composition, or with other therapeutic agents, or administration of the same or different agents by a different method, or administration as part of a different drug delivery device than a subject composition. The comparison may be made against the same or a different effective dosage of the various agents.

Alternatively, a comparison of the different treatment regimens described above may be based on the effectiveness of the treatment, using standard indices known to those of skill in the art. One method of treatment may be 10%, 20%, 30%, 50%, 75%, 100%, 150%, 200%, 300% more effective, than another method.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using the same or different therapeutic agents.

Kits

This invention also provides kits for conveniently and effectively implementing the methods of this invention. Such kits comprise any subject composition, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

### EXEMPLIFICATION

### Example 1

70 mg of DPPC and 28 mg of cholesterol were dissolved in 1 mL of ethanol and added to 10 mL of 4 mg/mL cisplatin in 0.9% saline solution. An aliquot (50%) of the sample was treated by 3 cycles of cooling to 4° C. and warming to 50° C. The aliquot, in a test tube, was cooled by refrigeration, and heated in a water bath. The resulting unentrapped cisplatin (free cisplatin) was washed by dialysis. The remainder of the sample was not treated by temperature cycles and directly washed by dialysis. Table 1 presents the percentage entrapment of cisplatin with and without cooling an warming cycles.

TABLE 1
Cisplatin percentage entrapment.

| | Final Concentration of | |
|---|---|---|
| | cisplatin, μg/ml | % Entrapment |
| Lipid-complexed cisplatin | 56 | 1.4 |
| without cooling and | | |
| warming cycles | | |
| Lipid-complexed cisplatin | 360 | 9.0 |
| after cooling and warming | | |
| cycles | | |

### Example 2

1.0 g of DPPC and 0.4 g of cholesterol were dissolved in 6 mL of ethanol. 60 mg of cisplatin was dissolved in 10 mL of 0.9% saline solution at 65° C. 1 mL of the resultant lipid mixture solution was added to 10 mL of the resultant cisplatin solution. The lipid/cisplatin suspension was cooled to approximately 4° C. and held at that temperature for 20 minutes and warmed to 50° C. and held at that temperature for 20 minutes. Ethanol was removed by bubbling N₂ gas into the suspension during the warming period. The cooling and warming steps were repeated 5 further times. The concentration of total cisplatin was 5.8 mg/mL with 91.6% entrapped cisplatin and drug: lipid ratio (by weight) of 1:26.

### Example 3

A liposomal formulation was prepared using phosphatidylcholine (PC) and cholesterol (in a 57:43 mol ratio). 0.55 mmoles of PC and 0.41 mmoles of cholesterol were dissolved in 2 mL ethanol and added to 20 mL of 4 mg/mL cisplatin solution. An aliquot (50%) of each sample was treated by 3 cycles of cooling and warming and then washed by dialysis. Another part of each sample was directly washed by dialysis. Entrapment was estimated from the ratio of final concentration and initial concentration.

TABLE 2
Entrapment and drug to lipid ratios for cisplatin
with various phophatidylcholines.

| | No Cooling and Warming | | | Cooling and Warming | | |
|---|---|---|---|---|---|---|
| | Final | | | Final | | |
| | [Cisplatin] | % | Drug:Lipid | [Cisplatin] | % | Drug:Lipid |
| PC | (mg/mL) | Entrapment | (by weight) | (mg/mL) | Entrapment | (by weight) |
| DOPC | 0.16 | 4.0 | 1:142 | 0.21 | 5.3 | 1:108 |
| EggPC | 0.09 | 2.3 | 1:247 | 0.12 | 3.0 | 1:185 |
| DMPC | 0.15 | 3.8 | 1:123 | 0.24 | 6.0 | 1:77 |
| DPPC | 0.17 | 4.3 | 1:115 | 0.85 | 21.3 | 1:23 |
| HSPC | 0.11 | 2.8 | 1:202 | 0.23 | 5.8 | 1:97 |
| DSPC | 0.10 | 2.5 | 1:184 | 0.58 | 14.5 | 1:32 |

### Example 4

A lipid formulation (DPPC:cholesterol in a ratio of 5:2 w/w) was dissolved in ethanol and added to a cisplatin solution. Part of the formulation was treated by cycles of cooling to 4° C. and warming to 55° C. cycles while part was not treated thus. The lipid/cisplatin suspension was then washed by dialysis.

TABLE 3
Concentration of cisplatin with and without cooling and warming cycles.

| Starting | | | |
|---|---|---|---|
| concentration of | Concentration of | Cooling | Total concentration |
| Cisplatin solution | lipids | & warming | of Cisplatin |
| (mg/mL) | (mg/mL) | cycles | (mg/mL) |
| 0.2 | 1.4 | No | Not Detectable |
| 0.2 | 1.4 | Yes | Not Detectable |
| 4.0 | 28 | No | 0.22 |
| 4.0 | 28 | Yes | 0.46 |

### Example 5

Dosing Schedule

Patients are dosed with a jet nebulizer (Pari LC Star) which is filled with up to about 7 mL of the lipid composition (containing about 1 mg/mL of cisplatin) which is formulated with saline. The flow rate of the lipid composition from the nebulizer is about 0.2 mL/min. At this rate, for example, administration of about 4 mL of the lipid composition takes about 20 minutes. Table 4 indicates the dosing schedule.

TABLE 4
Dosing schedule.

| | | Frequency of | |
|---|---|---|---|
| | Dose/Treatment Cycle | Treatment Cycles | # of Treatment |
| Patient | (mg/m²) | (week(s)) | Cycles |
| 1 | 1.5 | 3 | 6 (i.e., 18 weeks) |
| 2 | 3.0 | 3 | 6 |
| 3 | 6.0 | 3 | 6 |
| 4 | 12.0 | 3 | 6 |
| 5 | 24.0 | 3 | 6 |
| 6 | 48.0 | 3 | 6 |
| 7 | 24.0 | 2 | 6 (i.e., 12 weeks) |
| 8 | 36.0 | 2 | 6 |
| 9 | 48.0 | 2 | 6 |
| 10 | 24.0 | 1 | 12 (i.e., 3 months) |
| 11 | 36.0 | 2 | 3 |
| 12 | 24.0 | 2 | 4 |
| 13 | 36.0 | 2 | 2 |
| 14 | 36.0 | 2 | 4 |
| 15 | 48.0 | 2 | 4 |
| 16 | 60.0 | 2 | 2 |
| 17 | 60.0 | 2 | 1 |
| 18 | 80.0 | 2 | 1.5 |

Table 5 comprises the results of the study.

TABLE 5
Patient Results.

| | Initial Cisplatin Dose Level (mg/m²) | | | | | | | | | |
|---|---|---|---|---|---|---|---|---|---|---|
| Best | | | | | | | | | | |
| Overall Response¹ | 1.5 | 3.0 | 6.0 | 12.0 | 24.0 | 36.0 | 48.0 | 60.0 | 80.0 | Overall |
| Number of | 1 | 1 | 1 | 1 | 4 | 4 | 3 | 2 | 1 | 18 |
| Patients | | | | | | | | | | |
| Stable | 1 | 0 | 1 | 0 | 3 | 3 | 2 | 2 | 1 | 13 |
| Disease | | | | | | | | | | |
| Progressive | 0 | 1 | 0 | 1 | 1 | 1 | 1 | 0 | 0 | 5 |
| Disease | | | | | | | | | | |

¹RECIST Criteria.

Patient numbers 1, 3, 5, 6, 7, 9, 10, 11, 13, 14, 16, 17, and 18 of the ongoing study have shown stabilization (i.e., no further tumor growth or tumor growth of less than 20%).

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein.

We claim:

1. A method of treating lung diseases or pre-lung disease conditions in a subject in need thereof comprising administering to the subject a lipid formulation comprising an anticancer agent, wherein
a) the dose amount of anticancer agent is in the range of 1.5 mg/m² to 80 mg/m², and
b) the frequency of treatment cycles is no greater than 3 weeks.

2. The method of claim 1, wherein the anticancer agent is a platinum compound.

3. The method of claim 1, wherein the anticancer agent is cisplatin.

4. The method of claim 1, wherein the lipid formulation comprises a phospholipid.

5. The method of claim 1, wherein the lipid formulation comprises a steroid.

6. The method of claim 1, wherein the lipid formulation comprises a sterol.

7. The method of claim 1, wherein the lipid formulation comprises a phospholipid and a sterol.

8. The method of claim 1, wherein the lipid formulation comprises dipalmitoylphosphatidylcholine (DPPC) and cholesterol.

9. The method of claim 1, wherein the frequency of treatment cycles is no greater than 2 weeks.

10. The method of claim 1, wherein the frequency of treatment cycles is no greater than 1 week.

11. The method of claim 1, wherein the treatment is daily for anywhere from 1 to 7 days.

12. The method of claim 1, wherein the dose amount of the anticancer agent is in the range of 3.0, 6.0, 12.0, 24.0, 30.0, 36.0, 40.0, 48.0, or 60.0 mg/m² to 80 mg/m².

13. The method of claim 1, wherein the lipid formulation is administered by inhalation.

14. The method of claim 1, wherein the lipid formulation is administered intraperitonealy.

15. The method of claim 1, wherein the lipid formulation is administered intravenously.

16. The method claim 1, wherein the anticancer agent is a platinum compound, and the liposome formulation comprises a phospholipid and a sterol.

17. The method of claim 1, wherein the anticancer agent is a platinum compound, and the frequency of treatment cycles is no greater than 2 weeks.

18. The method of claim 1, wherein the anticancer agent is a platinum compound, and the frequency of treatment cycles is no greater than 1 week.

19. The method of claim 1, wherein the anticancer agent is a platinum compound and the dose amount of the platinum compound is in the range of 3.0, 6.0, 12.0, 24.0, 30.0, 36.0, 40.0, 48.0, or 60.0 mg/m² to 80 mg/m².

20. The method of claim 1, wherein the anticancer agent is cisplatin and the lipid formulation comprises a phospholipid and a sterol.

21. The method of claim 1, wherein the anticancer agent is cisplatin and the lipid formulation comprises DPPC and cholesterol.

22. The method of claim 1, wherein the anticancer agent is cisplatin and the frequency of treatment cycles is no greater than 2 weeks.

23. The method of claim 1, wherein the anticancer agent is cisplatin and the frequency of treatment cycles is no greater than 1 week.

24. The method of claim 1, wherein the anticancer agent is cisplatin and the dose amount of the cisplatin is in the range of 3.0, 6.0, 12.0, 24.0, 30.0, 36.0, 40.0, 48.0, or 60.0 mg/m to 80 mg/m².

25. The method of claim 1, wherein the anticancer agent is cisplatin, the lipid formulation comprises DPPC and cholesterol, and the frequency of treatment cycles is no greater than 2 weeks.

26. The method of claim 1, wherein the anticancer agent is cisplatin, the lipid formulation comprises DPPC and sterol, the frequency of treatment cycles is no greater than 2 weeks, and the dose amount of the cisplatin is in the range of 3.0, 6.0, 12.0, 24.0, 30.0, 36.0, 40.0, 48.0, or 60.0 mg/m² to 80 mg/m².

27. The method of claim 1, wherein the anticancer agent is cisplatin, the lipid formulation comprises DPPC and sterol, and the frequency of treatment cycles is no greater than 1 week.

28. The method of claim 1, wherein the anticancer agent is cisplatin, the lipid formulation comprises DPPC and sterol, the frequency of treatment cycles is no greater than 1 week, and the dose amount of the cisplatin is in the range of 3.0, 6.0, 12.0, 24.0, 30.0, 36.0, 40.0, 48.0, or 60 mg/m² to 80 mg/m².

29. The method of claim 1, wherein the anticancer agent is cisplatin, the lipid formulation comprises DPPC and cholesterol, the frequency of treatment cycles is no greater than 2 weeks, and the dose amount of the cisplatin is in the range of 3.0, 6.0, 12.0, 24.0, 30.0, 36.0, 40.0, 48.0, or 60.0 mg/m² to 80 mg/m².

30. The method of claim 1, wherein the anticancer agent is cisplatin, the lipid formulation comprises DPPC and sterol, the frequency of treatment cycles is no greater than 1 week.

31. The method of claim 1, wherein the anticancer agent is cisplatin, the lipid formulation comprises DPPC and cholesterol, the frequency of treatment cycles is no greater than 1 week, and the dose amount of the cisplatin is in the range of 3.0, 6.0, 12.0, 24.0, 30.0, 36.0, 40.0, 48.0, or 60.0 mg/m² to 80 mg/m².

32. A system for treating lung diseases or pre-lung disease conditions in a subject in need thereof comprising:
a) a lipid formulation comprising an anticancer agent, wherein the dose amount of anticancer agent is in the range of 1.5 mg/m² to 80 mg/m², and the frequency of treatment cycles is no greater than 3 weeks; and
b) an inhalation device.

33. The system of claim 32, wherein the inhalation device is disposable.
